(19) United States
(12) Patent Application Publication
(10) Pub. No.: US 2025/0134944 A1
(43) Pub. Date: May 1, 2025

(54) TEA FRACTION FOR TREATING IDIOPATHIC PULMONARY FIBROSIS

(71) Applicant: GT COMFORT HEALTH CORP., Taipei City (TW)

(72) Inventors: Jir-Mehng LO, Hsinchu County (TW); Cheng HUANG, Taipei City (TW); Limei HUANG, Taipei City (TW)

(73) Assignee: GT COMFORT HEALTH CORP., Taipei City (TW)

(21) Appl. No.: 18/883,956

(22) Filed: Sep. 12, 2024

Related U.S. Application Data
(60) Provisional application No. 63/593,345, filed on Oct. 26, 2023.

(51) Int. Cl. A61K 36/82; A61P 11/00; A61P 31/14

(52) U.S. Cl. CPC A61K 36/82; A61P 11/00; A61P 31/14; A61K 2236/33; A61K 2236/331; A61K 2236/39; A61K 2236/51

(57) ABSTRACT

The present invention provides a new tea fraction, which is made from the non-ethanol extract, and does not contain caffeine, epigallocatechin gallate (EGCG), gallic acid and catechin that are usually found in conventional tea extracts. The tea fraction has an efficacy in treating a lung fibrosis, particularly idiopathic pulmonary fibrosis (IPF), Covid-19 and long Covid.

### CROSS REFERENCE

This application claims priority to U.S. Provisional Patent Appl. No. 63/593,345 filed on Oct. 26, 2023, the contents of which is incorporated by reference in its entirety.

### FIELD OF THE INVENTION

The present invention relates to a new fraction and a new approach for treating idiopathic pulmonary fibrosis (IPF). In particular, the present invention relates to a new tea fraction effective for treating IPF, Covid-19 and long-Covid.

### BACKGROUND OF THE INVENTION

Idiopathic pulmonary fibrosis (IPF) is a progressive inflammatory disorder driven by a fibrotic cascade in the lungs in a sequential manner. IPF incidences were raising rapidly across the world. Recently, most severe COVID-19 patients develop hyper-inflammation and pneumonia. The major risk factors for severe COVID-19 are shared with IPF, namely increasing age, male sex, and comorbidities such as hypertension and diabetes. However, the role of antifibrotic therapy in patients with IPF is poorly defined. US Food and Drug Administration (FDA) approved pirfenidone and nintedanib (tyrosine kinase inhibitors) are being used as a first-line treatment drugs for IPF, however, neither the quality of life nor survival rates have been improved because of multiple side effects. Thus, the development of novel therapeutic approaches targeting transforming growth factor β (TGF-β) mediated cascade of fibrotic events is urgently needed to improve the survival of the patients.

Long Covid, also called “post Covid”, is a series of health problems persisting or developing after an initial Covid-19 infection. The symptoms can last for several weeks, months or even years. Long COVID is characterized by some symptoms, which sometimes disappear and reappear, including breathing discomfort, fatigue, cognitive dysfunction, memory problems, sleep disorder, headaches, loss of smell or taste, muscle weakness, fever and problems with mental health. The causes of long COVID are not yet fully understood. Breathing discomfort, fatigue, and cognitive dysfunction are three of the most prevalent. The respiratory symptoms of breathing discomfort are similar to those of IPF. It is believed that the approach for treating IPF is potential as that for treating breathing discomfort caused by long covid.

Accordingly, it is still desirable to develop a new approach to treat IPF, and breathing discomfort caused by covid-19 and long covid.

### BRIEF SUMMARY OF THE INVENTION

Accordingly, the present invention provides a new tea fraction enriching theabrownin, which is confirmed to be effective for treating a lung fibrosis, particularly idiopathic pulmonary fibrosis (IPF), and breathing discomfort, such as a symptom of IPF, covid-19 or long covid.

In one aspect, the present invention provides a tea fraction GT01-4, which is obtained by the method of the steps below:
(1) extracting tea leaves with ethanol to obtain an ethanol extract and residues of tea leaves;
(2) discarding the ethanol extract, and extracting the residues of tea leaves with water, to obtain an aqueous extract after filtration;
(3) allowing the aqueous extract to be partitioned with ethyl acetate (ETOAc) to obtain an aqueous extract; and
(4) concentrating and drying the aqueous extract to obtain a tea fraction GT01-4; wherein the tea fraction GT01-4 does not contain caffeine, epigallocatechin gallate (EGCG), gallic acid and catechin.

In the embodiments of the invention, the tea leaves are any kind of tea leaves, including green tea leaves, black tea leaves, pu-erh tea leaves and the like.

According to the invention, it is unexpectedly found that the tea fraction GT01-4 provides an efficacy in treating a lung fibrosis, particularly IPF.

According to the invention, it is also unexpectedly found that the tea fraction GT01-4 provides an anti-viral efficacy, particularly the efficacy in inhibition of SARS-CoV-2.

In one example of the invention, the tea fraction GT01-4 alleviates breathing discomfort in a subject suffering from a lung fibrosis, Covid-19 or long Covid. Accordingly, it is concluded that the tea fraction GT01-1 is potential to develop a medicament for treatment of Covid-19 or long Covid.

In another aspect, the present invention provides a composition or pharmaceutical composition for treating a lung fibrosis comprising a therapeutically effective amount of the tea fraction GT01-4, and a pharmaceutically acceptable carrier.

In one particular example of the invention, the lung fibrosis is idiopathic pulmonary fibrosis (IPF).

In a further aspect, the present invention provides a composition or pharmaceutical composition for treating Covid-19 or long Covid comprising a therapeutically effective amount of the tea fraction GT01-4, and a pharmaceutically acceptable carrier.

In a yet aspect, the present invention provides a method for treating a lung fibrosis, particularly IPF, in a subject, comprising administering to the subject the tea fraction GT01-4 in a therapeutically effective amount.

In a further yet aspect, the present invention provides a method for treating Covid-19 or long Covid in a subject, which comprises administering said subject a therapeutically effective amount of the tea fraction GT01-4.

In a yet further aspect, the present invention provides a use of the tea fraction GT01-4 for manufacturing a medicament for treating a lung fibrosis, particularly IPF, Covid-19 or long Covid.

It is to be understood that the foregoing general description and the following detailed description are exemplary and explanatory only and are not restrictive of the invention.

### BRIEF DESCRIPTION OF THE SEVERAL VIEWS OF THE DRAWINGS

The drawings presenting the preferred embodiments of the present invention are aimed at explaining the present invention. It should be understood that the present invention is not limited to the preferred embodiments shown.

FIG. 1A provides the protocol of the preparation of the tea fraction GT01-1.

FIG. 1B provides the protocol of the preparation of the tea fraction, GT01-2.

FIG. 1C provides the protocol of the preparation of the tea fraction GT01-3.

FIG. 1D provides the protocol of the preparation of the tea fraction GT01-4.

FIG. 2A provides the HPLC chromatograms of authentic caffeine, epigallocatechin gallate (EGCG), gallic acid and catechin by HPLC-UV (at 254 nm).

FIG. 2B provides the ELSD profile of the tea fraction GT01-4 as compared with the blank by ELSD (5-85% acn 65 min is C18 4.6*250 mm).

FIGS. 3A-3C show the anti-fibrotic activity of the four tea fractions (GT01-1, GT01-2, GT01-3 and GT01-4) and a positive control (EGCG) in TGF-β1-induced pulmonary fibrotic LL29 cells; wherein FIG. 3A shows the Western blotting of fibrotic biomarkers of each of the tea fractions GT01-1, GT01-2, GT01-3, GT01-4, and EGCG (a positive control); FIG. 3B shows the relative COL1A1/α-tubulin protein expression levels of the tea fractions GT01-1, GT01-2, GT01-3, GT01-4, and EGCG (a positive control); and FIG. 3C shows the relative α-SMA/α-tubulin protein expression levels of the tea fractions, GT01-1, GT01-2, GT01-3, GT01-4, and EGCG (a positive control). The protein levels relative to the vehicle control group were shown. Data are shown as the mean±SEM. One-way ANOVA with Dunnett's multiple comparisons test was performed to determine the statistical significance between the group treated with 5 ng/ml TGF-β1 alone and the other groups, where the significant increase (asterisks) and decrease (hashes) are indicated (e.g., * p<0.05; ** p<0.01; and *** p<0.001).

FIG. 4A-D show the inhibitory effect on the SARS-CoV-2 3CLpro activity of each of the tea fractions: GT01-1, GT01-2, GT01-3, GT01-4. After the incubation of the tea fractions with 0.25 μM SARS-CoV-2 3CLpro for an hour at 37° C. before adding 5 μM SARS-CoV-2 3CLprospecific IQF peptide, it was found that the efficacy of the tea fraction GT01-4 was the best among the four fractions. Seven or eight data points were obtained and shown as the mean±SEM.

FIG. 5 shows the inhibitory effect of the tea fraction GT01-4 on HBV replication in HepG2.2.15 cells; wherein the cells and the GT01-4 were incubated at the concentration as indicated for 48 hours, the amounts of HBsAg in the conditioned medium were determined by ELISA, where the negative control (NC) group was set to 0.

### DETAILED DESCRIPTION OF THE INVENTION

Unless otherwise defined herein, scientific and technical terms used herein have the meanings that are commonly understood by those of ordinary skill in the art.

As used herein, the singular forms “a”, “an”, and “the” include plural referents unless the context clearly dictates otherwise. Thus, for example, reference to “a sample” includes a plurality of such samples and equivalents thereto known to those skilled in the art.

According to the invention, a tea fraction GT01-4 is provided. The GT01-4 is obtained by the method of the steps below:
(1) extracting tea leaves with ethanol to obtain an ethanol extract and residues of tea leaves;
(2) discarding the ethanol extract, and extracting the residues of tea leaves with water, to obtain an aqueous extract after filtration;
(3) allowing the aqueous extract to be partitioned with ethyl acetate (ETOAc) to obtain an aqueous extract; and
(4) concentrating and drying the aqueous extract to obtain a tea fraction GT01-4; wherein the tea fraction GT01-4 does not contain caffeine, epigallocatechin gallate (EGCG), gallic acid and catechin; and is effective in treating idiopathic pulmonary fibrosis (IPF).

According to the invention, are any kind of tea leaves can be used to obtain the tea fraction GT01-4, including green tea leaves, black tea leaves, pu-erh tea leaves and the like.

In the example of the invention, it is ascertained that the tea fraction GT01-4 provides an efficacy in treating IPF. In addition, the tea fraction GT01-4 is also confirmed to provide an anti-viral efficacy, particularly the efficacy in inhibition of SARS-CoV-2. It was found that the tea fraction GT01-4 alleviated breathing discomfort in a subject suffering from a lung fibrosis, Covid-19 or long Covid. It is believed that the tea fraction GT01-1 is potential to develop a medicament for treatment of Covid-19 or long Covid.

For use in therapy, the therapeutically effective amount of the tea fraction GT01-4 is formulated as a pharmaceutical composition for administration, particularly oral administration. Accordingly, the invention further provides a pharmaceutical composition comprising a therapeutically effective amount of the tea fraction GT01-4, and one or more pharmaceutically acceptable carriers.

The term “subject” as used herein includes a human and/or a non-human animal, such as companion animals (e.g., dogs, cats, etc.), farm animals (e.g. cattle, sheep, pigs, horses, etc.), or experimental animals (e.g., rats, mice, guinea pigs, etc.).

The term “treat,” “treating” or “treatment” as used herein refers to the application or administration of a composition including one or more active agents to a subject afflicted with a disease, a symptom or conditions of the disease, or a progression of the disease, with the purpose to cure, heal, alleviate, relieve, alter, remedy, ameliorate, improve, or affect the disease, the symptoms or conditions of the disease, the disabilities induced by the disease, or the progression of the disease.

The term “therapeutically effective amount” as used herein refers to an amount of a pharmaceutical agent which, as compared to a corresponding subject who has not received such amount, results in an effect in treatment, healing, prevention, or amelioration of a disease, disorder, or side effect, or a decrease in the rate of advancement of a disease or disorder. The term also includes within its scope amounts effective to enhance normal physiological function.

The term “pharmaceutically acceptable carrier” used herein refers to a carrier(s), diluent(s) or excipient(s) that is acceptable, in the sense of being compatible with the other ingredients of the formulation and not deleterious to the subject to be administered with the pharmaceutical composition. Any carrier, diluent or excipient commonly known or used in the field may be used in the invention, depending to the requirements of the pharmaceutical formulation. Said carrier may be a diluent, vehicle, excipient, or matrix to the active ingredient. Some examples of appropriate excipients include lactose, dextrose, sucrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents.

The present invention is further illustrated by the following examples, which are provided for the purpose of demonstration rather than limitation.

### EXAMPLE

### Example 1 Preparation for the Tea Fractions According to the Invention

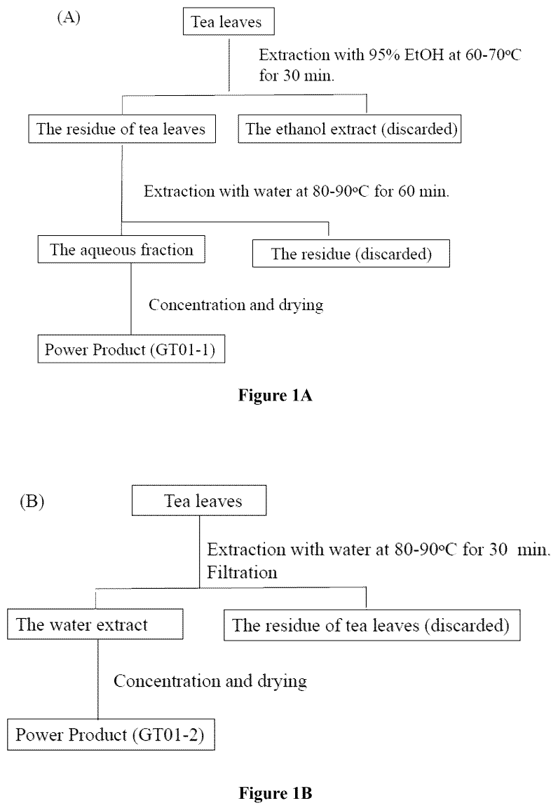

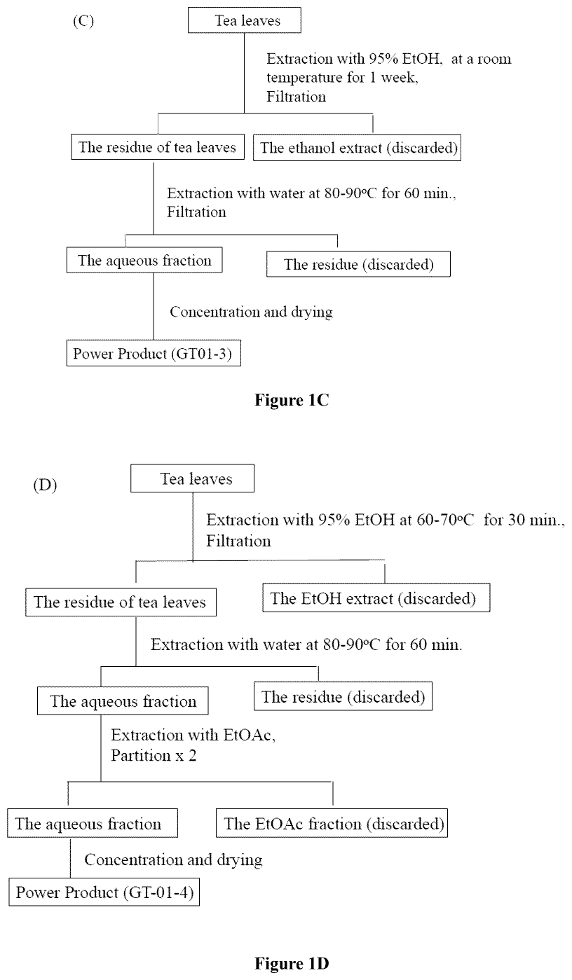

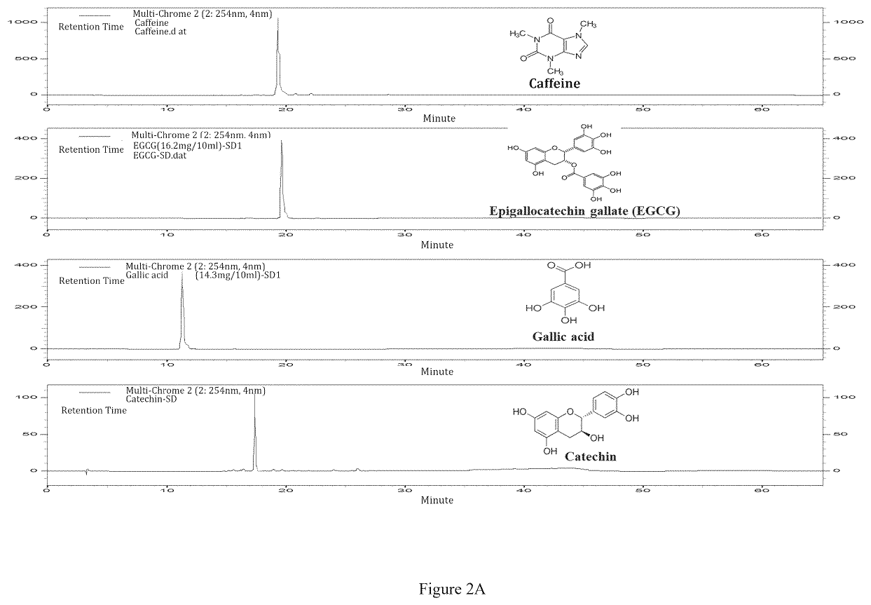

The four tea fractions including GT01-1, GT01-2, GT01-3 and GT01-4 were prepared according to the protocols shown in FIG. 1, which are briefly described below.

### Preparation of the Fraction GT01-1

As shown in FIG. 1(A), tea leaves (10 g) were extracted with 95% ethanol (500 ml) for 30 min. at 60-70° C., and the ethanol extract was discarded. The residue of tea leaves was extracted with distilled water for 60 min. at 80-90° C. and filtered. The resulting aqueous extract was concentrated and lyophilized to obtain the fraction GT01-1.

### Preparation of the Fraction GT01-2

As shown in FIG. 1(B), tea leaves (10 g) were extracted with distilled water (500 ml) for 30 min. at 80-90° C., and filtered. The residue of tea leaves was discarded and the resulting aqueous extract was concentrated and lyophilized to obtain the fraction GT01-2.

### Preparation of the Fraction GT01-3

As shown in FIG. 1(C), tea leaves (10 g) were extracted with 95% ethanol (500 ml) for one week at a room temperature, and the ethanol extract was discarded. The residue of tea leaves was extracted with distilled water (500 ml) for 60 min. at 80-90° C. and filtered. The resulting aqueous extract was concentrated and lyophilized to obtain the fraction GT01-3.

### Preparation of the Fraction GT01-4

As shown in FIG. 1(C), tea leaves (10 g) were extracted with 95% ethanol (500 ml) for 30 min. at 60-70° C., and the ethanol extract was discarded. The residue of tea leaves was extracted with distilled water (500 ml) for 60 min. at 80-90° C. and filtered. The resulting aqueous extract was partitioned with ethyl acetate (EtOAc) (500 ml) twice, and then the EtOAc fraction was discarded, and the water fraction was concentrated and lyophilized to obtain the tea fraction GT01-4.

### Characterization

The HPLC analysis was performed by a HPLC analyzer under the conditions below:
HPLC analysis conditions:
Column: ODS 3V
Mobile phase: CH₃OH/H₂O (0.1% H₃PO₄)
Flow rate: 1.0 ml/min.

| | | | | | |
|---|---|---|---|---|---|
| Time (min.) | 0 | 30 | 40 | 55 | 65 |
| CH₃OH | 5 | 70 | 85 | 5 | 5 |
| H₂O (0.1% H₃PO₄) | 95 | 30 | 15 | 95 | 95 |

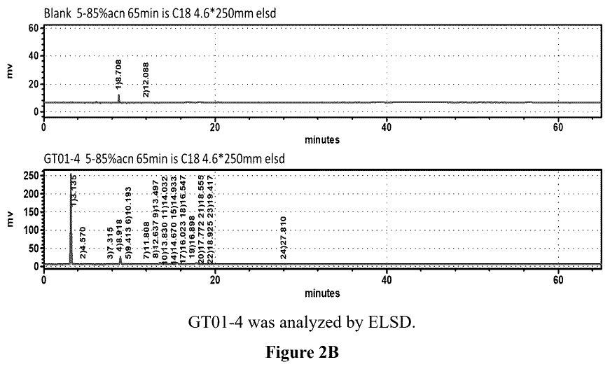

As shown in FIG. 2(A), Caffeine, EGCG, gallic acid and catechin are usually present in conventional tea extracts, which were analyzed by a HPLC-UV. As shown in FIG. 2(B), the tea fraction GT01-4 was analyzed by an evaporative light scattering detector (ELSD) and found a peak at RT 3.135 min. (5-85% acn 65 min is C18 4.6*250 mm ELSD). ELSD is a detector which is used in conjunction with high-performance liquid chromatography (HPLC), or ultra high-performance liquid chromatography (UHPLC), purification liquid chromatograph such as flash or preparative chromatography, countercurrent or centrifugal partition chromatography and supercritical fluid chromatography (SFC). It is commonly used for analysis of compounds where UV detection might be a restriction and therefore used where compounds do not efficiently absorb UV radiation, such as sugars, antivirals, antibiotics, fatty acids, lipids, oils, phospholipids, polymers, surfactants, terpenoids and triglycerides. An evaporative light scattering detector (ELSD) is able to detect all compound which are less volatile than the mobile phase, i.e. non-volatile and semi-volatile compounds.

Unexpectedly, it was found by the ELSD analysis that the tea fraction GT01-4 does not contain caffeine, EGCG, gallic acid and catechin that are usually found in conventional tea extracts.

### Example 2 Test for Efficacy in Treating Lung Fibroblast

### Cell Lines

LL 29 cells are human, lung fibroblasts derived from lung tissue with idiopathic pulmonary fibrosis. LL29 cells were purchased from ATCC®. Cells were maintained in Dulbecco's modified Eagle's medium (Gibco, USA) with 10% FBS (Gibco, USA) at 37° C. in an atmosphere of >95% humidity and 5% CO2 and were passaged every 2-3 days.

### Western Blot Analysis

Cell lysates were prepared in RIPA buffer. For western blot analysis, equal amount of the lysate samples were resolved by SDS-PAGE, followed by transfer to PVDF membranes, following the protocol published previously (Chiang et al., 2020). Antibodies against COL1A1 and α-SMA were purchased from GeneTex, Taiwan. HRP conjugated secondary antibodies were from Jackson ImmunoResearch Laboratories, Inc., USA. WesternBright® ECL kits were used for protein visualization (Advansta Inc., USA). Levels of protein expression were quantified in Fiji (Schindelin et al., 2012).

### Statistical Analysis

Data (N=3) were analyzed and plotted with GraphPad Prism 7.03 (GraphPad, USA). Values were expressed as the mean±standard error mean (SEM). Student's t-tests were performed to determine the statistical significance between the two groups.

One-way ANOVA post hoc Dunnett's multiple comparison tests were used to determine the statistical significance between the control group and two or more treated groups. Statistical significance was denoted by asterisks or hashtags (e.g., *, p<0.05; **, p<0.01; ***, p<0.001).

### Results

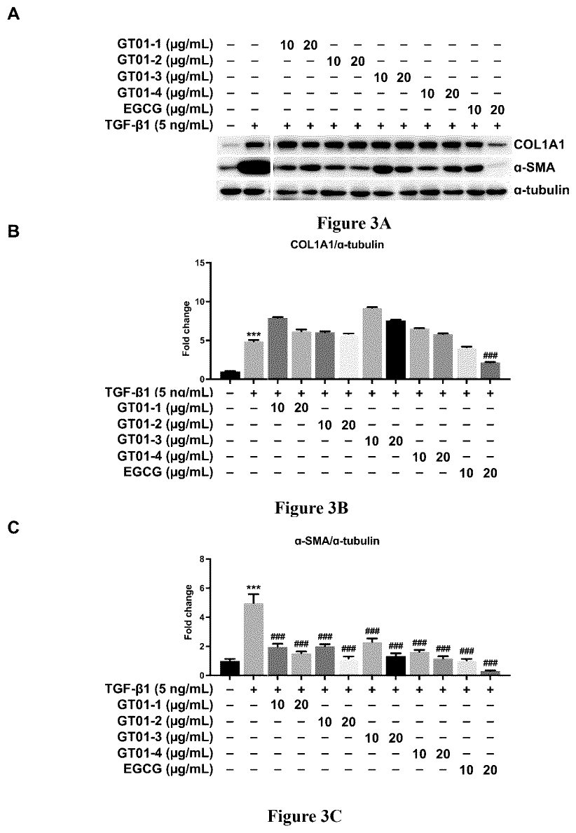

To determine the anti-fibrotic activity of the test compounds against pulmonary fibrosis, human lung fibroblasts LL29 cells were treated with 5 ng/ml TGF-β1 and each fraction at the concentration as indicated was incubated for 48 hours. The densitometry analysis was performed to quantify the protein levels of the western blot 17 in Fiji. As shown in FIG. 3(A), the TGF-β1 treatment alone increased the protein levels of COL1A1 anda-SMA. Meanwhile, the treatment of each of the four fractions led to changes in the COL1A1 and α-SMA levels to various extents. As shown in FIG. 3(B), the four tea fractions led a decrease in the COL1A1 protein level as compared with the group treated with TGF-β1 alone. As shown in FIG. 3(C), all of the four tea fractions diminished the α-SMA expression substantially, and the tea fraction GT01-4 provided a significant decrease in the α-SMA protein level. In view of the above, the tea fraction GT01-4 showed the activity in improving the α-SMA level in TGF-β1-induced pulmonary fibrotic LL29 cells, and was potential on ameliorating lung fibrosis.

### Example 3 Test for Efficacy in Inhibition of SARS-CoV-2

### Materials and Methods

FRET Protease Assays with the SARS-CoV-2 3CLpro

The establishment of an ED-FRET platform follows the protocol given by Jo et al. (2020). Briefly, a custom proteolytic, fluorogenic peptide with DABCYL and EDANS on ends, DABCYL-TSAVLQSGFRKMG-EDANS (Genomics, Taiwan), contains the consensus nsp4/nsp5 cleavage sequence that can be recognized by 3CLpro of SARSCoV-2. The peptide is dissolved in distilled water and incubated with 3CLpro of SARS-CoV-2. Measurements of the spectral-based fluorescence are determined by a SPARK® multimode microplate reader provided by TECAN. The proteolytic activity is determined at 37° C. by fluorescent intensity of EDANS upon peptide hydrolysis as a function of time, in which λexcitation=340 nm, λemission=490 nm, bandwidths=9, 15 nm, respectively. Prior to the assay, the emission wavelength of the testing drugs at 340 nm excitation is examined to ensure that it does not overlap with the emission spectrum of EDANS. Assays are conducted in triplicate in black 96-well microplates (Greiner) in 100 μL assay buffers containing 3CLpro of SARS-CoV-2 and the customized peptide. In SARS 3CLpro assay, 0.25 μM SARS-CoV-2 3CLpro containing 50 mM Tris pH 6.5 is incubated with 5 μM fluorescent substrate at 37° C. for 3 h before measuring Relative Fluorescence Unit (RFU). Inhibition assays for the four fractions were performed. At first, the SARS-CoV-2 3CLpro and the four fractions were mixed and pre-incubated at 37° C. for 1 hour respectively. The manifesting inhibitory activity against 3CLpro of SARS-CoV-2 for each group was investigated at different concentrations to characterize their IC50 values, using GraphPad Prism 7.03 (GraphPad Software, San Diego, CA, USA).

### Results

Based the knowledge and sequence-based SARS-CoV-2 3CLpro, the efficacy of 3CLpro inhibiting the four fractions were assessed in vitro to determine their therapeutic potential in SARS-CoV-2 treatment. Until now, developing a broad-spectrum antiviral agent to combat against SARS-CoV-2 is of utmost importance and urgency. Enactment of ED-FRET technology and its workflow provides a robust, high-throughput drug discovery in the lab. Meanwhile, identification of SARS-CoV-2 3CLpro inhibiting agents from the tea fractions proposed and provided the activities as the guidelines of probable therapeutic doses in clinical assessment.

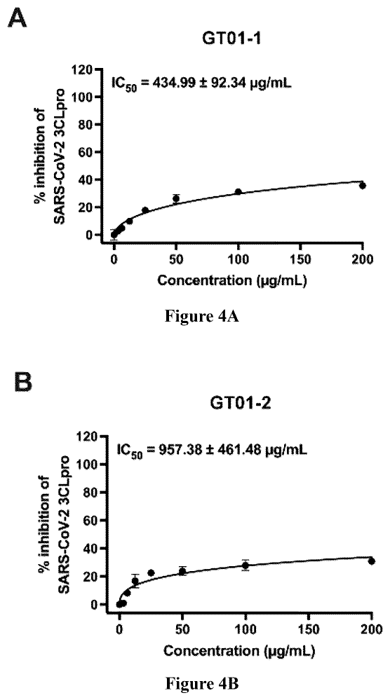

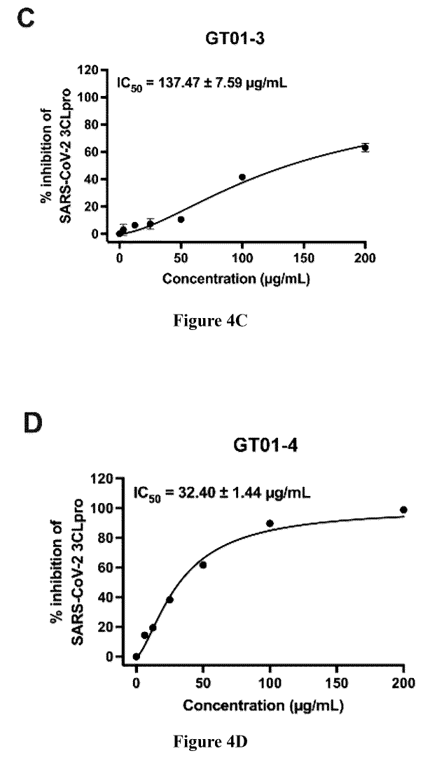

To determine the inhibitory effect of the tea fractions on the SARS-CoV-2 3CLpro activity, protease activity assays of SARS-CoV-2 3CLpro were performed. As shown in FIG. 4, the IC50 values of GT01-1 (FIG. 4(A)) and GT01-2 (FIG. 4(B)) against SARS-CoV-2 3CLpro were estimated to be about 434.99±92.34 μg/mL and 957.38±461.48 μg/mL, respectively. As for GT01-3 (FIG. 4(C)), it had an IC50 value of 137.47±7.59 μg/mL against SARS-CoV-2 3CLpro. GT01-4 (FIG. 4(D)) at 32.40±1.44 μg/mL, manifested 50% inhibition of the SARS-CoV-2 3CLpro activity. Specifically, 25, 50, 100, and 200 μg/mL GT01-4 inhibited the SARS-CoV-2 3CLpro activity by about 38%, 62%, 90%, and 99%, respectively. Taken together, the tea fraction GT01-4 inhibited SARS-CoV-2 3CLpro more potently than other three tea fractions.

### Example 4 Test for Anti-Viral Efficacy

### Cell Lines

### HepG2.2.15 Cells-Cell Model

HepG2.2.15 cells are used because of the unlimited supply and constant quality and were maintained in Dulbecco's modified Eagle medium (DMEM; Invitrogen) supplemented with 10% heat-inactivated fetal bovine serum (FBS; Thermo) plus 100 units of penicillin and 100 μg of streptomycin per ml (both from Invitrogen). To test the effect of the tea fraction GT01-4 on HBV replication. Each The tea fraction GT01-4 was added to the medium at two concentrations and cultured for 48 hours, when the HBV were collected from supernatant. The ELISA assay was used to detect HBV HBsAg as index of efficiency of HBV replication.

The experimental methods are as followed Enzyme-linked immunosorbent assay (ELISA): The HBsAg ELISA Kit (General Biologicals Corp.) are used to detect hepatitis B surface antigen (HBsAg) with the protocol suggested.

### Statistical Analysis

All values are expressed as mean±SE. Each value is the mean of at least three experiments in each drugs in vitro experiments. Student's t-test is used for statistical comparison. * indicates that the values are significantly different from the control (*, p<0.05; **, P<0.01; ***, P<0.001.).

### Results

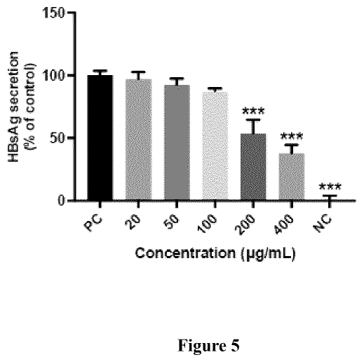

To determine the anti-HBV activity of test compounds, HepG2.2.15 cells were incubated with a test compound for 48 hours, after which the HBsAg level in the conditioned medium was determined by ELISA. As shown in FIG. 5, GT01-4 manifested a significant reduction of the HBsAg secretion at the two highest concentrations tested, down to about 54% and 38%. It is confirmed that the GT01-4 had an anti-viral efficacy and is potential on inhibition of virus infection.

While the present invention has been disclosed by way preferred embodiments, it is not intended to limit the present invention. Any person of ordinary skill in the art may, without departing from the spirit and scope of the present invention, shall be allowed to perform modification and embellishment. Therefore, the scope of protection of the present invention shall be governed by which defined by the claims attached subsequently.

### REFERENCE

1. Chen, T., Nie, H., Gao, X., Yang, J., Pu, J., Chen, Z., . . . . Jia, G. (2014). Epithelialmesenchymal transition involved in pulmonary fibrosis induced by multiwalled carbon nanotubes via TGF-beta/Smad signaling pathway. Toxicol Lett, 226(2), 150-162.
2. Chiang, H., Lee, J. C., Huang, H. C., Huang, H., Liu, H. K., & Huang, C. (2020). Delayed intervention with a novel SGLT2 inhibitor NGI001 suppresses dietinduced metabolic dysfunction and non-alcoholic fatty liver disease in mice. Br J Pharmacol, 177(2), 239-253.
3. Kim, H. S., Yoon, Y. M., Meang, M. K., Park, Y. E., Lee, J. Y., Lee, T. H., Youn, B. S. (2019). Reversion of in vivo fibrogenesis by novel chromone scaffolds. EBioMedicine, 39, 484-496.
4. Molina-Molina, M., Machahua-Huamani, C., Vicens-Zygmunt, V., Llatjos, R., Escobar, I., Sala-Llinas, E., Montes-Worboys, A. (2018). Anti-fibrotic effects of pirfenidone and rapamycin in primary IPF fibroblasts and human alveolar epithelial cells. BMC Pulm Med, 18(1), 63.
5. Schindelin, J., Arganda-Carreras, I., Frise, E., Kaynig, V., Longair, M., Pietzsch, T., Cardona, A. (2012). Fiji: an open-source platform for biological-image analysis. Nat Methods, 9(7), 676-682.

What is claimed is:

1. A tea fraction GT01-4, which is obtained by the method of the steps below:
(1) extracting tea leaves with ethanol to obtain an ethanol extract and residues of tea leaves;
(2) discarding the ethanol extract, and extracting the residues of tea leaves with water, to obtain an aqueous extract after filtration;
(3) allowing the aqueous extract to be partitioned with ethyl acetate (ETOAc) to obtain an aqueous extract; and
(4) concentrating and drying the aqueous extract to obtain a tea fraction GT01-4;
wherein the tea fraction GT01-4 does not contain caffeine, epigallocatechin gallate (EGCG), gallic acid and catechin.

2. The tea fraction GT01-4 of claim 1, wherein the tea leaves are selected from the group consisting of green tea leaves, black tea leaves, pu-erh tea leaves and combination thereof.

3. The tea fraction GT01-4 of claim 1, which provides an efficacy in treating a lung fibrosis.

4. The tea fraction GT01-4 of claim 3, in which the lung fibrosis is idiopathic pulmonary fibrosis (IPF).

5. The tea fraction GT01-4 of claim 1, which provides anti-viral efficacy.

6. The tea fraction GT01-4 of claim 5, which provides an efficacy in inhibition of SARS-CoV-2.

7. The tea fraction GT01-4 of claim 6, which provides an efficacy in treating Covid-19 or long Covid.

8. The tea fraction GT01-4 of claim 1, which alleviates breathing discomfort in a subject suffering from a lung fibrosis, Covid-19 or long Covid.

9. A composition or pharmaceutical composition for treating a lung fibrosis comprising a therapeutically effective amount of the tea fraction GT01-4 as defined in claim 1, and a pharmaceutically acceptable carrier.

10. The pharmaceutical composition of claim 9, wherein the lung fibrosis is idiopathic pulmonary fibrosis (IPF).

11. A composition or pharmaceutical composition for treating Covid-19 or long Covid comprising a therapeutically effective amount of the tea fraction GT01-4 as defined in claim 1, and a pharmaceutically acceptable carrier.

12. A method for treating a lung fibrosis in a subject, comprising administering to the subject the tea fraction GF01-4 in a therapeutically effective amount.

13. The method of claim 12, wherein the lung fibrosis is idiopathic pulmonary fibrosis (IPF).

14. A method for treating Covid-19 or long Covid in a subject, comprising administering to the subject the tea fraction according to the invention in a therapeutically effective amount.

15. A method for alleviating breathing discomfort in a subject suffering from a lung fibrosis, Covid-19 or long Covid, comprising administering to the subject the tea fraction according to the invention in a therapeutically effective amount or the composition thereof.
